(19) United States
(12) Patent Application Publication
(10) Pub. No.: US 2015/0111237 A1
(43) Pub. Date: Apr. 23, 2015

(54) BIOTINIDASE ASSAYS

(71) Applicant: ADVANCED LIQUID LOGIC, INC., San Diego, CA (US)

(72) Inventors: Carrie A. Graham, Raleigh, NC (US); Allen E. Eckhardt, San Diego, CA (US)

(73) Assignee: ADVANCED LIQUID LOGIC, INC., San Diego, CA (US)

(21) Appl. No.: 14/399,051

(22) PCT Filed: May 7, 2013
(86) PCT No.: PCT/US2013/039867

§ 371 Date: Nov. 5, 2014

Related U.S. Application Data
(60) Provisional application No. 61/643,711, filed on May 7, 2012.

(51) Int. Cl. C12Q 1/34

(52) U.S. Cl. CPC C12Q 1/34
USPC 435/18

(57) ABSTRACT

The invention relates to methods for enzymatic detection of biotinidase activity. In certain aspects, the invention provides methods for bench-based enzymatic detection of biotinidase activity. In other aspects, the invention provides methods for bench-based enzymatic detection in droplets in oil. The droplet-based methods may be performed in an automated manner using digital microfluidics technology on a droplet actuator device. The enzymatic assays for biotinidase activity may be used for newborn testing for biotinidase deficiency, and may be combined with other droplet-based enzymatic assays in a panel of tests for newborn testing.

### RELATED APPLICATIONS

In addition to the patent applications cited herein, each of which is incorporated herein by reference, this patent application is related to and claims priority to U.S. Provisional Patent Application No. 61/643,711, filed on May 7, 2012, entitled “Biotinidase Assays,” the entire disclosure of which is incorporated herein by reference.

### GOVERNMENT INTEREST

This invention was made with government support under HD057713 awarded by National Institutes of Health. The United States Government has certain rights in the invention.

### BACKGROUND OF THE INVENTION

A droplet actuator typically includes one or more substrates configured to form a surface or gap for conducting droplet operations. The one or more substrates establish a droplet operations surface or gap for conducting droplet operations and may also include electrodes arranged to conduct the droplet operations. The droplet operations substrate or the gap between the substrates may be coated or filled with a filler fluid that is immiscible with the liquid that forms the droplets.

Droplet actuators are used in a variety of applications, including molecular diagnostic assays such as enzymatic assays. In one application, enzymatic assays are used as part of a routine testing process to test newborn infants for various genetic disorders. For example, enzymatic assays may be used to test for deficiencies in biotinidase activity. Currently, many laboratories screen for biotinidase deficiency using a qualitative fluorescent spot test that requires visual inspection, which may be subjective. Therefore, there is a need for new approaches to biotinidase deficiency testing that is less prone to error and that is more sensitive and reliable.

### SUMMARY OF THE INVENTION

The invention provides a method of conducting an assay for biotinidase. The method includes, among other things, providing a sample, providing a substrate formulation, wherein the substrate formulation comprises a substrate that releases a fluorophore upon contact with biotinidase, mixing the sample with the substrate formulation to produce a reaction sample, incubating the reaction sample, mixing a stop buffer with the reaction sample, and measuring a fluorescence signal in the reaction sample, wherein the fluorescence signal correlates with the presence and/or activity of biotinidase in the sample. The enzymatic assays for biotinidase activity may be used for newborn testing for biotinidase deficiency, and may be combined with other droplet-based enzymatic assays in a panel of tests for newborn testing.

In one embodiment, the biotinidase assay method of the invention may be performed with a sample that includes fresh blood, fresh-frozen blood, plasma, fresh-frozen plasma, or a dried blood spot. The sample may be from a newborn infant. The fresh blood, fresh-frozen blood, plasma, fresh-frozen plasma, or dried blood spot may be mixed with an extraction buffer. In extraction buffer may comprise a surfactant, including a polysorbate surfactant such as Polysorbate 20.

In another embodiment, the biotinidase assay method of the invention may be performed with a substrate formulation that further comprises an assay buffer. The assay buffer may include a pH buffer, a reducing agent, and a surfactant. The pH buffer for the assay buffer may be a potassium phosphate buffer, and may have a pH of about 6.5. The reducing agent for the assay buffer may be dithiothreitol (DTT). The surfactant for the assay buffer may be a polysorbate surfactant such as Polysorbate 20.

In yet another embodiment of the biotinidase assay method of the invention, the substrate that releases a fluorophore upon contact with biotinidase is a biotin-4-MU fluorescent substrate. The biotin-4-MU fluorescent substrate may be n-D-biotinyl-7-amino-4-methylcoumarin, and the step of measuring a fluorescence signal may include reading fluorescence at 360 nm excitation and 460 nm emission. The biotin-4-MU fluorescent substrate may be dissolved in a solvent before addition to the substrate formulation. The solvent may be a polar aprotic solvent such as dimethyl sulfoxide (DMSO).

In another embodiment of the biotinidase assay method of the invention, the reaction sample is incubated at between 36° C. and 38° C., particularly at about 37° C. The reaction sample may be incubated for up to 24 hours, up to 20 hours, or up to 1 hour.

In yet another embodiment of the biotinidase assay method of the invention, the stop buffer may include a pH buffer and a surfactant. The pH buffer may be a sodium bicarbonate buffer, and may have a pH of between 10.0 and 11.0. The surfactant for the stop buffer may be a polysorbate surfactant such as Polysorbate 20.

In another embodiment of the biotinidase assay method of the invention, any of the steps of the method may be performed in one or more droplets in oil, such as silicone oil.

In yet another embodiment of the biotinidase assay method of the invention, the sample may be loaded onto a droplet actuator and the method may be performed by executing droplet operations on the droplet actuator. The droplet actuator may comprise a filler fluid comprising an oil, such as a silicone oil. The filler fluid further may also include a nonionic low hydrophile-lipophile balanced (HLB) surfactant. The droplet operations may be executed by the droplet actuator using include electrode-mediated droplet operations, electrowetting mediated droplet operations, or dielectrophoresis mediated droplet operations.

In another embodiment, the invention provides a computer readable medium programmed to cause a droplet actuator to perform any of the method steps of the biotinidase assay method of the invention. The invention also provides a system comprising a droplet actuator coupled to and controlled by a computer programmed to cause the droplet actuator to perform any of the method steps of the biotinidase assay method of the invention.

Further features and other aspects of the invention are more clearly evident from the following detailed discussion and from the appended claims.

### DEFINITIONS

As used herein, the following terms have the meanings indicated.

“Activate,” with reference to one or more electrodes, means affecting a change in the electrical state of the one or more electrodes which, in the presence of a droplet, results in a droplet operation. Activation of an electrode can be accomplished using alternating or direct current. Any suitable voltage may be used. For example, an electrode may be activated using a voltage which is greater than about 150 V, or greater than about 200 V, or greater than about 250 V, or from about 275 V to about 375 V, or about 300 V. Where alternating current is used, any suitable frequency may be employed. For example, an electrode may be activated using alternating current having a frequency from about 1 Hz to about 100 Hz, or from about 10 Hz to about 60 Hz, or from about 20 Hz to about 40 Hz, or about 30 Hz.

“Bead,” with respect to beads on a droplet actuator, means any bead or particle that is capable of interacting with a droplet on or in proximity with a droplet actuator. Beads may be any of a wide variety of shapes, such as spherical, generally spherical, egg shaped, disc shaped, cubical, amorphous and other three dimensional shapes. The bead may, for example, be capable of being subjected to a droplet operation in a droplet on a droplet actuator or otherwise configured with respect to a droplet actuator in a manner which permits a droplet on the droplet actuator to be brought into contact with the bead on the droplet actuator and/or off the droplet actuator. Beads may be provided in a droplet, in a droplet operations gap, or on a droplet operations surface. Beads may be provided in a reservoir that is external to a droplet operations gap or situated apart from a droplet operations surface, and the reservoir may be associated with a flow path that permits a droplet including the beads to be brought into a droplet operations gap or into contact with a droplet operations surface. Beads may be manufactured using a wide variety of materials, including for example, resins, and polymers. The beads may be any suitable size, including for example, microbeads, microparticles, nanobeads and nanoparticles. In some cases, beads are magnetically responsive; in other cases beads are not significantly magnetically responsive. For magnetically responsive beads, the magnetically responsive material may constitute substantially all of a bead, a portion of a bead, or only one component of a bead. The remainder of the bead may include, among other things, polymeric material, coatings, and moieties which permit attachment of an assay reagent. Examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® particles, available from Invitrogen Group, Carlsbad, Calif.), fluorescent microparticles and nanoparticles, coated magnetic microparticles and nanoparticles, ferromagnetic microparticles and nanoparticles, coated ferromagnetic microparticles and nanoparticles, and those described in U.S. Patent Publication Nos. 20050260686, entitled “Multiplex flow assays preferably with magnetic particles as solid phase,” published on Nov. 24, 2005; 20030132538, entitled “Encapsulation of discrete quanta of fluorescent particles,” published on Jul. 17, 2003; 20050118574, entitled “Multiplexed Analysis of Clinical Specimens Apparatus and Method,” published on Jun. 2, 2005; 20050277197. Entitled “Microparticles with Multiple Fluorescent Signals and Methods of Using Same,” published on Dec. 15, 2005; 20060159962, entitled “Magnetic Microspheres for use in Fluorescence-based Applications,” published on Jul. 20, 2006; the entire disclosures of which are incorporated herein by reference for their teaching concerning beads and magnetically responsive materials and beads. Beads may be pre-coupled with a biomolecule or other substance that is able to bind to and form a complex with a biomolecule. Beads may be pre-coupled with an antibody, protein or antigen, DNA/RNA probe or any other molecule with an affinity for a desired target. Examples of droplet actuator techniques for immobilizing magnetically responsive beads and/or non-magnetically responsive beads and/or conducting droplet operations protocols using beads are described in U.S. patent application Ser. No. 11/639,566, entitled “Droplet-Based Particle Sorting,” filed on Dec. 15, 2006; U.S. Patent Application No. 61/039,183, entitled “Multiplexing Bead Detection in a Single Droplet,” filed on Mar. 25, 2008; U.S. Patent Application No. 61/047,789, entitled “Droplet Actuator Devices and Droplet Operations Using Beads,” filed on Apr. 25, 2008; U.S. Patent Application No. 61/086,183, entitled “Droplet Actuator Devices and Methods for Manipulating Beads,” filed on Aug. 5, 2008; International Patent Application No. PCT/US2008/053545, entitled “Droplet Actuator Devices and Methods Employing Magnetic Beads,” filed on Feb. 11, 2008; International Patent Application No. PCT/US2008/058018, entitled “Bead-based Multiplexed Analytical Methods and Instrumentation,” filed on Mar. 24, 2008; International Patent Application No. PCT/US2008/058047, “Bead Sorting on a Droplet Actuator,” filed on Mar. 23, 2008; and International Patent Application No. PCT/US2006/047486, entitled “Droplet-based Biochemistry,” filed on Dec. 11, 2006; the entire disclosures of which are incorporated herein by reference. Bead characteristics may be employed in the multiplexing aspects of the invention. Examples of beads having characteristics suitable for multiplexing, as well as methods of detecting and analyzing signals emitted from such beads, may be found in U.S. Patent Publication No. 20080305481, entitled “Systems and Methods for Multiplex Analysis of PCR in Real Time,” published on Dec. 11, 2008; U.S. Patent Publication No. 20080151240, “Methods and Systems for Dynamic Range Expansion,” published on Jun. 26, 2008; U.S. Patent Publication No. 20070207513, entitled “Methods, Products, and Kits for Identifying an Analyte in a Sample,” published on Sep. 6, 2007; U.S. Patent Publication No. 20070064990, entitled “Methods and Systems for Image Data Processing,” published on Mar. 22, 2007; U.S. Patent Publication No. 20060159962, entitled “Magnetic Microspheres for use in Fluorescence-based Applications,” published on Jul. 20, 2006; U.S. Patent Publication No. 20050277197, entitled “Microparticles with Multiple Fluorescent Signals and Methods of Using Same,” published on Dec. 15, 2005; and U.S. Patent Publication No. 20050118574, entitled “Multiplexed Analysis of Clinical Specimens Apparatus and Method,” published on Jun. 2, 2005.

“Droplet” means a volume of liquid on a droplet actuator. Typically, a droplet is at least partially bounded by a filler fluid. For example, a droplet may be completely surrounded by a filler fluid or may be bounded by filler fluid and one or more surfaces of the droplet actuator. As another example, a droplet may be bounded by filler fluid, one or more surfaces of the droplet actuator, and/or the atmosphere. As yet another example, a droplet may be bounded by filler fluid and the atmosphere. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, combinations of such shapes, and various shapes formed during droplet operations, such as merging or splitting or formed as a result of contact of such shapes with one or more surfaces of a droplet actuator. For examples of droplet fluids that may be subjected to droplet operations using the approach of the invention, see International Patent Application No. PCT/US 06/47486, entitled, “Droplet-Based Biochemistry,” filed on Dec. 11, 2006. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. Moreover, a droplet may include a reagent, such as water, deionized water, saline solutions, acidic solutions, basic solutions, detergent solutions and/or buffers. Other examples of droplet contents include reagents, such as a reagent for a biochemical protocol, such as a nucleic acid amplification protocol, an affinity-based assay protocol, an enzymatic assay protocol, a sequencing protocol, and/or a protocol for analyses of biological fluids. A droplet may include one or more beads.

“Droplet Actuator” means a device for manipulating droplets. For examples of droplet actuators, see Pamula et al., U.S. Pat. No. 6,911,132, entitled “Apparatus for Manipulating Droplets by Electrowetting-Based Techniques,” issued on Jun. 28, 2005; Pamula et al., U.S. patent application Ser. No. 11/343,284, entitled “Apparatuses and Methods for Manipulating Droplets on a Printed Circuit Board,” filed on filed on Jan. 30, 2006; Pollack et al., International Patent Application No. PCT/US2006/047486, entitled “Droplet-Based Biochemistry,” filed on Dec. 11, 2006; Shenderov, U.S. Pat. No. 6,773,566, entitled “Electrostatic Actuators for Microfluidics and Methods for Using Same,” issued on Aug. 10, 2004 and U.S. Pat. No. 6,565,727, entitled “Actuators for Microfluidics Without Moving Parts,” issued on Jan. 24, 2000; Kim and/or Shah et al., U.S. patent application Ser. No. 10/343,261, entitled “Electrowetting-driven Micropumping,” filed on Jan. 27, 2003, Ser. No. 11/275,668, entitled “Method and Apparatus for Promoting the Complete Transfer of Liquid Drops from a Nozzle,” filed on Jan. 23, 2006, Ser. No. 11/460,188, entitled “Small Object Moving on Printed Circuit Board,” filed on Jan. 23, 2006, Ser. No. 12/465,935, entitled “Method for Using Magnetic Particles in Droplet Microfluidics,” filed on May 14, 2009, and Ser. No. 12/513,157, entitled “Method and Apparatus for Real-time Feedback Control of Electrical Manipulation of Droplets on Chip,” filed on Apr. 30, 2009; Velev, U.S. Pat. No. 7,547,380, entitled “Droplet Transportation Devices and Methods Having a Fluid Surface,” issued on Jun. 16, 2009; Sterling et al., U.S. Pat. No. 7,163,612, entitled “Method, Apparatus and Article for Microfluidic Control via Electrowetting, for Chemical, Biochemical and Biological Assays and the Like,” issued on Jan. 16, 2007; Becker and Gascoyne et al., U.S. Pat. No. 7,641,779, entitled “Method and Apparatus for Programmable fluidic Processing,” issued on Jan. 5, 2010, and U.S. Pat. No. 6,977,033, entitled “Method and Apparatus for Programmable fluidic Processing,” issued on Dec. 20, 2005; Decre et al., U.S. Pat. No. 7,328,979, entitled “System for Manipulation of a Body of Fluid,” issued on Feb. 12, 2008; Yamakawa et al., U.S. Patent Pub. No. 20060039823, entitled “Chemical Analysis Apparatus,” published on Feb. 23, 2006; Wu, International Patent Pub. No. WO/2009/003184, entitled “Digital Microfluidics Based Apparatus for Heat-exchanging Chemical Processes,” published on Dec. 31, 2008; Fouillet et al., U.S. Patent Pub. No. 20090192044, entitled “Electrode Addressing Method,” published on Jul. 30, 2009; Fouillet et al., U.S. Pat. No. 7,052,244, entitled “Device for Displacement of Small Liquid Volumes Along a Micro-catenary Line by Electrostatic Forces,” issued on May 30, 2006; Marchand et al., U.S. Patent Pub. No. 20080124252, entitled “Droplet Microreactor,” published on May 29, 2008; Adachi et al., U.S. Patent Pub. No. 20090321262, entitled “Liquid Transfer Device,” published on Dec. 31, 2009; Roux et al., U.S. Patent Pub. No. 20050179746, entitled “Device for Controlling the Displacement of a Drop Between two or Several Solid Substrates,” published on Aug. 18, 2005; Dhindsa et al., “Virtual Electrowetting Channels: Electronic Liquid Transport with Continuous Channel Functionality,” Lab Chip, 10:832-836 (2010); the entire disclosures of which are incorporated herein by reference, along with their priority documents. Certain droplet actuators will include one or more substrates arranged with a droplet operations gap therebetween and electrodes associated with (e.g., layered on, attached to, and/or embedded in) the one or more substrates and arranged to conduct one or more droplet operations. For example, certain droplet actuators will include a base (or bottom) substrate, droplet operations electrodes associated with the substrate, one or more dielectric layers atop the substrate and/or electrodes, and optionally one or more hydrophobic layers atop the substrate, dielectric layers and/or the electrodes forming a droplet operations surface. A top substrate may also be provided, which is separated from the droplet operations surface by a gap, commonly referred to as a droplet operations gap. Various electrode arrangements on the top and/or bottom substrates are discussed in the above-referenced patents and applications and certain novel electrode arrangements are discussed in the description of the invention. During droplet operations it is preferred that droplets remain in continuous contact, frequent contact or intermittent contact with a ground or reference electrode. A ground or reference electrode may be associated with the top substrate facing the gap, the bottom substrate facing the gap, in the gap. Where electrodes are provided on both substrates, electrical contacts for coupling the electrodes to a droplet actuator instrument for controlling or monitoring the electrodes may be associated with one or both plates. In some cases, electrodes on one substrate are electrically coupled to the other substrate so that only one substrate is in contact with the droplet actuator. In one embodiment, a conductive material (e.g., an epoxy, such as MASTER BOND™ Polymer System EP79, available from Master Bond, Inc., Hackensack, N.J.) provides the electrical connection between electrodes on one substrate and electrical paths on the other substrates, e.g., a ground electrode on a top substrate may be coupled to an electrical path on a bottom substrate by such a conductive material. Where multiple substrates are used, a spacer may be provided between the substrates to determine the height of the gap therebetween and define dispensing reservoirs. The spacer height may, for example, be from about 5 μm to about 600 μm, or about 100 μm to about 400 μm, or about 200 μm to about 350 μm, or about 250 μm to about 300 μm, or about 275 μm. The spacer may, for example, be formed of a layer of projections form the top or bottom substrates, and/or a material inserted between the top and bottom substrates. One or more openings may be provided in the one or more substrates for forming a fluid path through which liquid may be delivered into the droplet operations gap. The one or more openings may in some cases be aligned for interaction with one or more electrodes, e.g., aligned such that liquid flowed through the opening will come into sufficient proximity with one or more droplet operations electrodes to permit a droplet operation to be effected by the droplet operations electrodes using the liquid. The one or more openings may in some cases serve as vents for releasing liquid or gas from within the droplet operations gap. In some cases, the openings may be sealed or covered with a permeable material such as a membrane. For example a membrane having oleophobicity and hydrophobicity, such as VERSAPOR® Membrane (Pall Corp., Port Washington, N.Y.) may be used to cover an opening to facilitate escape of gasses while preventing escape of oil and aqueous liquids. The base (or bottom) and top substrates may in some cases be formed as one integral component, such as a folded or layered plastic or layered semiconductor construction. One or more reference electrodes may be provided on the base (or bottom) and/or top substrates and/or in the gap. Examples of reference electrode arrangements are provided in the above referenced patents and patent applications. In various embodiments, the manipulation of droplets by a droplet actuator may be electrode mediated, e.g., electrowetting mediated or dielectrophoresis mediated or Coulombic force mediated. Examples of other techniques for controlling droplet operations that may be used in the droplet actuators of the invention include using devices that induce hydrodynamic fluidic pressure, such as those that operate on the basis of mechanical principles (e.g. external syringe pumps, pneumatic membrane pumps, vibrating membrane pumps, vacuum devices, centrifugal forces, piezoelectric/ultrasonic pumps and acoustic forces); electrical or magnetic principles (e.g. electroosmotic flow, electrokinetic pumps, ferrofluidic plugs, electrohydrodynamic pumps, attraction or repulsion using magnetic forces and magnetohydrodynamic pumps); thermodynamic principles (e.g. gas bubble generation/phase-change-induced volume expansion); other kinds of surface-wetting principles (e.g. electrowetting, and optoelectrowetting, as well as chemically, thermally, structurally and radioactively induced surface-tension gradients); gravity; surface tension (e.g., capillary action); electrostatic forces (e.g., electroosmotic flow); centrifugal flow (substrate disposed on a compact disc and rotated); magnetic forces (e.g., oscillating ions causes flow); magnetohydrodynamic forces; and vacuum or pressure differential. In certain embodiments, combinations of two or more of the foregoing techniques may be employed to conduct a droplet operation in a droplet actuator of the invention. Similarly, one or more of the foregoing may be used to deliver liquid into a droplet operations gap, e.g., from a reservoir in another device or from an external reservoir of the droplet actuator (e.g., a reservoir associated with a droplet actuator substrate and a flow path from the reservoir into the droplet operations gap). Droplet operations surfaces of certain droplet actuators of the invention may be made from hydrophobic materials or may be coated or treated to make them hydrophobic. For example, in some cases some portion or all of the droplet operations surfaces may be derivatized with low surface-energy materials or chemistries, e.g., by deposition or using in situ synthesis using compounds such as poly- or per-fluorinated compounds in solution or polymerizable monomers. Examples include TEFLON® AF (available from DuPont, Wilmington, Del.), members of the cytop family of materials, coatings in the FLUOROPEL® family of hydrophobic and superhydrophobic coatings (available from Cytonix Corporation, Beltsville, Md.), silane coatings, fluorosilane coatings, hydrophobic phosphonate derivatives (e.g., those sold by Aculon, Inc), and NOVEC™ electronic coatings (available from 3M Company, St. Paul, Minn.), and other fluorinated monomers for plasma-enhanced chemical vapor deposition (PECVD). In some cases, the droplet operations surface may include a hydrophobic coating having a thickness ranging from about 10 nm to about 1,000 nm. Moreover, in some embodiments, the top substrate of the droplet actuator includes an electrically conducting organic polymer, which is then coated with a hydrophobic coating or otherwise treated to make the droplet operations surface hydrophobic. For example, the electrically conducting organic polymer that is deposited onto a plastic substrate may be poly(3,4-ethylenedioxythiophene) poly(styrenesulfonate) (PEDOT:PSS). Other examples of electrically conducting organic polymers and alternative conductive layers are described in Pollack et al., International Patent Application No. PCT/US2010/040705, entitled “Droplet Actuator Devices and Methods,” the entire disclosure of which is incorporated herein by reference. One or both substrates may be fabricated using a printed circuit board (PCB), glass, indium tin oxide (ITO)-coated glass, and/or semiconductor materials as the substrate. When the substrate is ITO-coated glass, the ITO coating is preferably a thickness in the range of about 20 to about 200 nm, preferably about 50 to about 150 nm, or about 75 to about 125 nm, or about 100 nm. In some cases, the top and/or bottom substrate includes a PCB substrate that is coated with a dielectric, such as a polyimide dielectric, which may in some cases also be coated or otherwise treated to make the droplet operations surface hydrophobic. When the substrate includes a PCB, the following materials are examples of suitable materials: MITSUI™ BN-300 (available from MITSUI Chemicals America, Inc., San Jose Calif.); ARLON™ 11N (available from Arlon, Inc, Santa Ana, Calif.).; NELCO® N4000-6 and N5000-30/32 (available from Park Electrochemical Corp., Melville, N.Y.); ISOLA™ FR406 (available from Isola Group, Chandler, Ariz.), especially IS620; fluoropolymer family (suitable for fluorescence detection since it has low background fluorescence); polyimide family; polyester; polyethylene naphthalate; polycarbonate; polyetheretherketone; liquid crystal polymer; cyclo-olefin copolymer (COC); cyclo-olefin polymer (COP); aramid; THERMOUNT® nonwoven aramid reinforcement (available from DuPont, Wilmington, Del.); NOMEX® brand fiber (available from DuPont, Wilmington, Del.); and paper. Various materials are also suitable for use as the dielectric component of the substrate. Examples include: vapor deposited dielectric, such as PARYLENE™ C (especially on glass) and PARYLENE™ N (available from Parylene Coating Services, Inc., Katy, Tex.); TEFLON® AF coatings; cytop; soldermasks, such as liquid photoimageable soldermasks (e.g., on PCB) like TAIYO™ PSR4000 series, TAIYO™ PSR and AUS series (available from Taiyo America, Inc. Carson City, Nev.) (good thermal characteristics for applications involving thermal control), and PROBIMER™ 8165 (good thermal characteristics for applications involving thermal control (available from Huntsman Advanced Materials Americas Inc., Los Angeles, Calif.); dry film soldermask, such as those in the VACREL® dry film soldermask line (available from DuPont, Wilmington, Del.); film dielectrics, such as polyimide film (e.g., KAPTON® polyimide film, available from DuPont, Wilmington, Del.), polyethylene, and fluoropolymers (e.g., FEP), polytetrafluoroethylene; polyester; polyethylene naphthalate; cyclo-olefin copolymer (COC); cyclo-olefin polymer (COP); any other PCB substrate material listed above; black matrix resin; and polypropylene. Droplet transport voltage and frequency may be selected for performance with reagents used in specific assay protocols. Design parameters may be varied, e.g., number and placement of on-actuator reservoirs, number of independent electrode connections, size (volume) of different reservoirs, placement of magnets/bead washing zones, electrode size, inter-electrode pitch, and gap height (between top and bottom substrates) may be varied for use with specific reagents, protocols, droplet volumes, etc. In some cases, a substrate of the invention may derivatized with low surface-energy materials or chemistries, e.g., using deposition or in situ synthesis using poly- or per-fluorinated compounds in solution or polymerizable monomers. Examples include TEFLON® AF coatings and FLUOROPEL® coatings for dip or spray coating, and other fluorinated monomers for plasma-enhanced chemical vapor deposition (PECVD). Additionally, in some cases, some portion or all of the droplet operations surface may be coated with a substance for reducing background noise, such as background fluorescence from a PCB substrate. For example, the noise-reducing coating may include a black matrix resin, such as the black matrix resins available from Toray industries, Inc., Japan. Electrodes of a droplet actuator are typically controlled by a controller or a processor, which is itself provided as part of a system, which may include processing functions as well as data and software storage and input and output capabilities. Reagents may be provided on the droplet actuator in the droplet operations gap or in a reservoir fluidly coupled to the droplet operations gap. The reagents may be in liquid form, e.g., droplets, or they may be provided in a reconstitutable form in the droplet operations gap or in a reservoir fluidly coupled to the droplet operations gap. Reconstitutable reagents may typically be combined with liquids for reconstitution. An example of reconstitutable reagents suitable for use with the invention includes those described in Meathrel, et al., U.S. Pat. No. 7,727,466, entitled “Disintegratable films for diagnostic devices,” granted on Jun. 1, 2010.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles. For examples of droplet operations, see the patents and patent applications cited above under the definition of “droplet actuator.” Impedance or capacitance sensing or imaging techniques may sometimes be used to determine or confirm the outcome of a droplet operation. Examples of such techniques are described in Sturmer et al., International Patent Pub. No. WO/2008/101194, entitled “Capacitance Detection in a Droplet Actuator,” published on Aug. 21, 2008, the entire disclosure of which is incorporated herein by reference. Generally speaking, the sensing or imaging techniques may be used to confirm the presence or absence of a droplet at a specific electrode. For example, the presence of a dispensed droplet at the destination electrode following a droplet dispensing operation confirms that the droplet dispensing operation was effective. Similarly, the presence of a droplet at a detection spot at an appropriate step in an assay protocol may confirm that a previous set of droplet operations has successfully produced a droplet for detection. Droplet transport time can be quite fast. For example, in various embodiments, transport of a droplet from one electrode to the next may exceed about 1 sec, or about 0.1 sec, or about 0.01 sec, or about 0.001 sec. In one embodiment, the electrode is operated in AC mode but is switched to DC mode for imaging. It is helpful for conducting droplet operations for the footprint area of droplet to be similar to electrowetting area; in other words, 1×-, 2×-3×-droplets are usefully controlled operated using 1, 2, and 3 electrodes, respectively. If the droplet footprint is greater than the number of electrodes available for conducting a droplet operation at a given time, the difference between the droplet size and the number of electrodes should typically not be greater than 1; in other words, a 2× droplet is usefully controlled using 1 electrode and a 3× droplet is usefully controlled using 2 electrodes. When droplets include beads, it is useful for droplet size to be equal to the number of electrodes controlling the droplet, e.g., transporting the droplet.

“Filler fluid” means a fluid associated with a droplet operations substrate of a droplet actuator, which fluid is sufficiently immiscible with a droplet phase to render the droplet phase subject to electrode-mediated droplet operations. For example, the droplet operations gap of a droplet actuator is typically filled with a filler fluid. The filler fluid may, for example, be a low-viscosity oil, such as silicone oil or hexadecane filler fluid. The filler fluid may fill the entire gap of the droplet actuator or may coat one or more surfaces of the droplet actuator. Filler fluids may be conductive or non-conductive. Filler fluids may, for example, be doped with surfactants or other additives. For example, additives may be selected to improve droplet operations and/or reduce loss of reagent or target substances from droplets, formation of microdroplets, cross contamination between droplets, contamination of droplet actuator surfaces, degradation of droplet actuator materials, etc. Composition of the filler fluid, including surfactant doping, may be selected for performance with reagents used in the specific assay protocols and effective interaction or non-interaction with droplet actuator materials. Examples of filler fluids and filler fluid formulations suitable for use with the invention are provided in Srinivasan et al, International Patent Pub. Nos. WO/2010/027894, entitled “Droplet Actuators, Modified Fluids and Methods,” published on Mar. 11, 2010, and WO/2009/021173, entitled “Use of Additives for Enhancing Droplet Operations,” published on Feb. 12, 2009; Sista et al., International Patent Pub. No. WO/2008/098236, entitled “Droplet Actuator Devices and Methods Employing Magnetic Beads,” published on Aug. 14, 2008; and Monroe et al., U.S. Patent Publication No. 20080283414, entitled “Electrowetting Devices,” filed on May 17, 2007; the entire disclosures of which are incorporated herein by reference, as well as the other patents and patent applications cited herein.

“Immobilize” with respect to magnetically responsive beads, means that the beads are substantially restrained in position in a droplet or in filler fluid on a droplet actuator. For example, in one embodiment, immobilized beads are sufficiently restrained in position in a droplet to permit execution of a droplet splitting operation, yielding one droplet with substantially all of the beads and one droplet substantially lacking in the beads.

“Magnetically responsive” means responsive to a magnetic field. “Magnetically responsive beads” include or are composed of magnetically responsive materials. Examples of magnetically responsive materials include paramagnetic materials, ferromagnetic materials, ferrimagnetic materials, and metamagnetic materials. Examples of suitable paramagnetic materials include iron, nickel, and cobalt, as well as metal oxides, such as Fe3O4, BaFe12O19, CoO, NiO, Mn2O3, Cr2O3, and CoMnP. “Reservoir” means an enclosure or partial enclosure configured for holding, storing, or supplying liquid. A droplet actuator system of the invention may include on-cartridge reservoirs and/or off-cartridge reservoirs. On-cartridge reservoirs may be (1) on-actuator reservoirs, which are reservoirs in the droplet operations gap or on the droplet operations surface; (2) off-actuator reservoirs, which are reservoirs on the droplet actuator cartridge, but outside the droplet operations gap, and not in contact with the droplet operations surface; or (3) hybrid reservoirs which have on-actuator regions and off-actuator regions. An example of an off-actuator reservoir is a reservoir in the top substrate. An off-actuator reservoir is typically in fluid communication with an opening or flow path arranged for flowing liquid from the off-actuator reservoir into the droplet operations gap, such as into an on-actuator reservoir. An off-cartridge reservoir may be a reservoir that is not part of the droplet actuator cartridge at all, but which flows liquid to some portion of the droplet actuator cartridge. For example, an off-cartridge reservoir may be part of a system or docking station to which the droplet actuator cartridge is coupled during operation. Similarly, an off-cartridge reservoir may be a reagent storage container or syringe which is used to force fluid into an on-cartridge reservoir or into a droplet operations gap. A system using an off-cartridge reservoir will typically include a fluid passage means whereby liquid may be transferred from the off-cartridge reservoir into an on-cartridge reservoir or into a droplet operations gap.

“Transporting into the magnetic field of a magnet,” “transporting towards a magnet,” and the like, as used herein to refer to droplets and/or magnetically responsive beads within droplets, is intended to refer to transporting into a region of a magnetic field capable of substantially attracting magnetically responsive beads in the droplet. Similarly, “transporting away from a magnet or magnetic field,” “transporting out of the magnetic field of a magnet,” and the like, as used herein to refer to droplets and/or magnetically responsive beads within droplets, is intended to refer to transporting away from a region of a magnetic field capable of substantially attracting magnetically responsive beads in the droplet, whether or not the droplet or magnetically responsive beads is completely removed from the magnetic field. It will be appreciated that in any of such cases described herein, the droplet may be transported towards or away from the desired region of the magnetic field, and/or the desired region of the magnetic field may be moved towards or away from the droplet. Reference to an electrode, a droplet, or magnetically responsive beads being “within” or “in” a magnetic field, or the like, is intended to describe a situation in which the electrode is situated in a manner which permits the electrode to transport a droplet into and/or away from a desired region of a magnetic field, or the droplet or magnetically responsive beads is/are situated in a desired region of the magnetic field, in each case where the magnetic field in the desired region is capable of substantially attracting any magnetically responsive beads in the droplet. Similarly, reference to an electrode, a droplet, or magnetically responsive beads being “outside of” or “away from” a magnetic field, and the like, is intended to describe a situation in which the electrode is situated in a manner which permits the electrode to transport a droplet away from a certain region of a magnetic field, or the droplet or magnetically responsive beads is/are situated away from a certain region of the magnetic field, in each case where the magnetic field in such region is not capable of substantially attracting any magnetically responsive beads in the droplet or in which any remaining attraction does not eliminate the effectiveness of droplet operations conducted in the region. In various aspects of the invention, a system, a droplet actuator, or another component of a system may include a magnet, such as one or more permanent magnets (e.g., a single cylindrical or bar magnet or an array of such magnets, such as a Halbach array) or an electromagnet or array of electromagnets, to form a magnetic field for interacting with magnetically responsive beads or other components on chip. Such interactions may, for example, include substantially immobilizing or restraining movement or flow of magnetically responsive beads during storage or in a droplet during a droplet operation or pulling magnetically responsive beads out of a droplet.

“Washing” with respect to washing a bead means reducing the amount and/or concentration of one or more substances in contact with the bead or exposed to the bead from a droplet in contact with the bead. The reduction in the amount and/or concentration of the substance may be partial, substantially complete, or even complete. The substance may be any of a wide variety of substances; examples include target substances for further analysis, and unwanted substances, such as components of a sample, contaminants, and/or excess reagent. In some embodiments, a washing operation begins with a starting droplet in contact with a magnetically responsive bead, where the droplet includes an initial amount and initial concentration of a substance. The washing operation may proceed using a variety of droplet operations. The washing operation may yield a droplet including the magnetically responsive bead, where the droplet has a total amount and/or concentration of the substance which is less than the initial amount and/or concentration of the substance. Examples of suitable washing techniques are described in Pamula et al., U.S. Pat. No. 7,439,014, entitled “Droplet-Based Surface Modification and Washing,” granted on Oct. 21, 2008, the entire disclosure of which is incorporated herein by reference. The terms “top,” “bottom,” “over,” “under,” and “on” are used throughout the description with reference to the relative positions of components of the droplet actuator, such as relative positions of top and bottom substrates of the droplet actuator. It will be appreciated that the droplet actuator is functional regardless of its orientation in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

### BRIEF DESCRIPTION OF THE DRAWINGS

FIGS. 1A and 1B show bar graphs of an example of biotinidase enzyme activity assays performed on-bench using DBS extract and serum samples;

FIG. 2 shows a data table of fluorescence readings for biotinidase assays performed on-bench using different concentrations of Tween® 20 (0.01% and 1.01%) and DTT (0.1 mM, 1.1 mM, and 10.1 mM);

FIG. 3 shows a bar graph of another example of a biotinidase enzyme activity assay performed on-bench using DBS extracts;

FIG. 4 shows a bar graph of yet another example of a biotinidase activity assay performed on-bench using DBS extracts;

FIG. 5 shows a plot of an example of a biotinidase activity assay for evaluating partitioning of 4-MU;

FIG. 6 shows a plot of an example of a biotinidase enzymatic assay via digital microfluidics on deficient and normal proficiency samples and confirmed affected and presumed normal patient samples; and

FIG. 7 illustrates a functional block diagram of an example of a microfluidics system that includes a droplet actuator.

### DETAILED DESCRIPTION OF THE INVENTION

The present invention provides methods for automated enzymatic detection of biotinidase activity. In one embodiment, the invention provides methods for enzymatic detection of biotinidase activity in droplets in oil. The droplet-based method includes, among other things, incubating a droplet in oil, the droplet comprising a substrate liquid and a sample liquid. In various embodiments, the invention includes methods for conducting biotinidase enzymatic activity assays in fresh blood samples, fresh-frozen blood samples, and dried blood spot (DBS) samples. In another embodiment, the invention provides methods for bench-based enzymatic detection of biotinidase activity.

In one example, the enzymatic assays for biotinidase activity may be used for newborn testing for biotinidase deficiency. Droplet-based enzymatic assays for biotinidase activity may be combined with other droplet-based enzymatic assays in a panel of tests for newborn testing.

### 7.1 Enzymatic Assays for Detection of Biotinidase Activity

Biotinidase deficiency is an inherited disorder of biotin recycling. When biotinidase activity is deficient, biotin can be neither removed from ingested food nor recycled from specific metabolic enzymes (e.g., carboxylases) to which it is bound. Biotin is covalently bound to carboxylase enzymes forming a complex called biocytin. Biotinidase removes biotin from biocytin and makes it available to be reused by other enzymes. Deficient biotinidase activity causes specific metabolic enzymes (i.e., carboxylases) to be nonfunctional, inhibiting the proper processing of proteins, fats, and carbohydrates.

In one example, the enzymatic assays of the invention may be performed in fresh or fresh-frozen whole blood samples or serum samples. An aliquot of fresh whole blood may be combined with an aliquot of extraction buffer such as 0.1% (w/v) Tween® 20 in molecular grade water and analyzed directly. Alternatively, the whole blood sample in extraction buffer may be stored at −80° C. until use. In one example, a 3.1 μL aliquot of whole blood may be diluted with 96.9 μL of extraction buffer (e.g., 0.1% (w/v) Tween® 20 in molecular grade water). The prepared blood sample may be assayed directly or stored at −80° C. until use.

In another example, the enzymatic assays of the invention may be performed in dried blood spot (DBS) extracts. DBS extracts may, for example, be prepared from blood samples collected and dried on filter paper. A manual or automatic puncher may be used to punch a sample, e.g., a 3 mm punch. Each punch may be placed into a separate well of a round bottomed 96-well plate. An aliquot (e.g., 100 μL) of extraction buffer such as 0.1% (w/v) Tween® 20 in molecular grade water may be added to each well that contains a DBS punch and incubated for about 30 minutes at 1600 rpm on a plate shaker at room temperature to extract the DBS samples. Extraction buffer composition (e.g., pH, detergent concentration, salts, etc.) may be selected for performance with reagents used in specific assay protocols. Where a dried blood spot is used, dried blood spots may be reconstituted in water, a saline solution, a buffer, and/or a solution including a surfactant. In one embodiment, the extraction buffer comprises a surfactant, including but not limited to a polysorbate surfactant such as Polysorbate 20 (Tween® 20).

Substrate formulations for the enzymatic assays of the invention may, for example, include an assay buffer (e.g., a pH buffer such as potassium phosphate buffer, between pH 6.0 to 7.0, particularly pH 6.5; a reducing agent such as dithiothreitol (DTT); and a surfactant, such as a polysorbate surfactant (e.g., Tween® 20), and a biotin-4-MU fluorescent substrate such as n-D-biotinyl-7-amino-4-methylcoumarin (B7A4MC). The biotin-4-MU substrate may be dissolved in an aliquot (e.g., 100 μL) of a solvent, for example a polar aprotic solvent such as dimethyl sulfoxide (DMSO), to improve solubility prior to mixing into the assay buffer and/or substrate formulation. Examples of fluorescent substrates, including 4-MU- or HMU containing substrates, are described in U.S. Pat. No. 8,394,641, entitled “Method of Hydrolyzing an Enzymatic Substrate,” issued Mar. 12, 2013.

The invention provides methods for droplet-based enzymatic detection of biotinidase activity and for bench-based enzymatic detection of biotinidase activity. In one example, the methods of the invention may include, but are not limited to, the following steps:
1. Preparing a sample, e.g. a blood sample;
2. Preparing a substrate formulation;
3. Mixing an aliquot of prepared sample (e.g., blood sample) with an aliquot of substrate formulation to produce a reaction sample;
4. Incubating the reaction sample at about 37° C.;
5. Stopping (quenching) the reaction after a sufficient period of incubation; and
6. Reading 4-MU fluorescence (e.g., 360 nm excitation/460 nm emission).

### 7.1.1 Assay Protocol

The invention provides methods for enzymatic detection of biotinidase activity. Biotinidase enzymatic activity assays may be performed using fresh blood samples, fresh-frozen blood samples, or dried blood spot (DBS) samples. The assay may be performed using a microtiter plate based assay and microtiter plate reader (e.g., Synergy H1 plate reader). The assay for biotinidase enzyme activity uses biotin-4-MU as substrate and detection of 4-MU fluorescence as output. The microtiter plate reader may be heated to an incubation temperature (e.g., 36° C. to 38° C., particularly 37° C.).

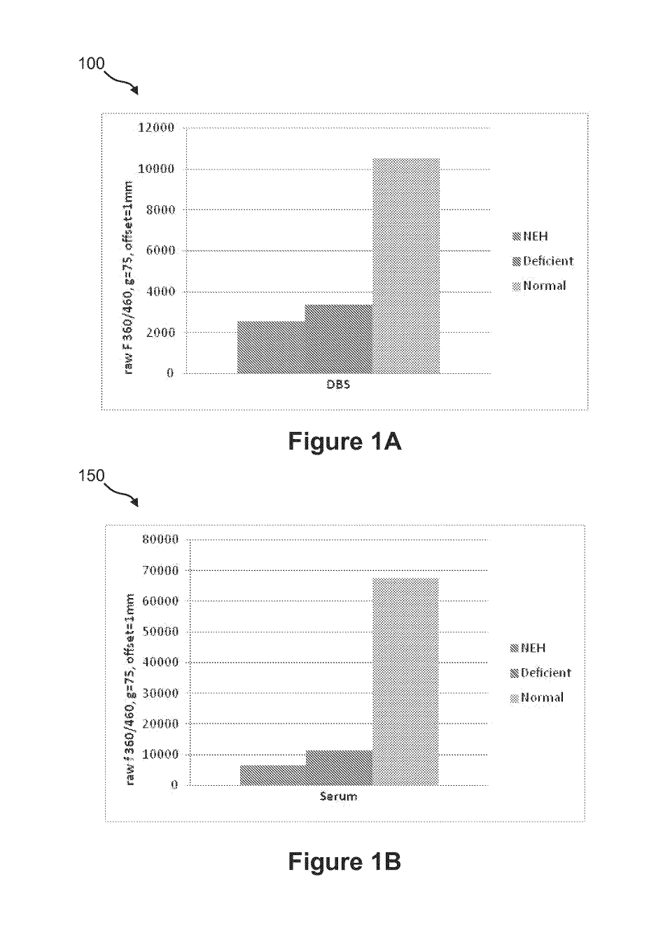

FIGS. 1A and 1B show bar graphs 100 and 150 of an example of biotinidase enzyme activity assays performed on-bench using DBS extract and serum samples, respectively. Fresh-frozen serum samples from a presumed normal individual and from a biotinidase “deficient” sample were used. “Deficient” serum samples were heat-treated prior to use to inactivate all biotinidase activity. DBS extract samples from a presumed normal individual and from a quality control (QC) base pool (BP) dried blood sample were prepared by extracting a 3 mm punch in 100 μL of extraction buffer (0.1% (w/v) Tween® 20 in molecular grade water) as described herein. The dried QC-BP sample was prepared from a pool of washed, leukoreduced human red blood cells that were adjusted with human plasma to a hematocrit of 50%. The substrate formulation was 0.15 M potassium phosphate, pH 6.5; 0.01% (w/v) Tween® 20; 0.1 mM DTT; and 0.2 mM biotin-4-MU (n-D-biotinyl-7-amino-4-methylcoumarin, obtained from Toronto Research Chemicals). The assay was performed on-bench using a microtiter plate assay and plate reader.

The assay protocol included the following steps: An aliquot (10 μL) of fresh-frozen serum sample or DBS extract was mixed with 10 μL of biotin-4-MU substrate mix in separate wells of a 96-well microtiter plate. The reaction was incubated at 37° C. for 20 hours. After the incubation period, the reaction was stopped with 50 μL stop (quench) buffer (0.2 M sodium bicarbonate, pH 10.0; 0.01% (w/v) Tween® 20). Fluorescence was read at 360 nm excitation/460 nm emission using a microtiter plate reader at a gain of 75, offset 1 mm. Samples were run in triplicate. To control for non-enzymatic hydrolysis (NEH) of the substrate, 10 μL of DBS extract and 10 μL of substrate mix were placed in separate wells of the 96-well microtiter plate. After the incubation period, the DBS extract and substrate mix was combined and the reaction stopped with 50 μL of stop (quench) buffer. Referring to bar graph 100 of FIG. 1A, the data shows the biotinidase activity in DBS extracts from a biotinidase deficient individual was well below and clearly separated from the biotinidase activity found in DBS extracts from a presumed normal individual. Referring to bar graph 150 of FIG. 1B, the data shows the biotinidase activity in serum from a biotinidase deficient individual was well below and clearly separated from the biotinidase activity found in serum from a presumed normal individual.

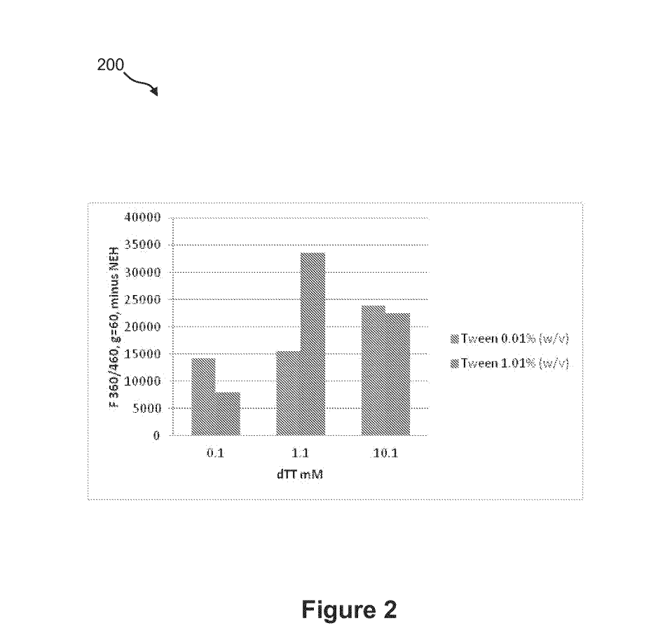

FIG. 2 shows a bar graph 200 of fluorescence readings for biotinidase assays performed on-bench using different concentrations of Tween® 20 (0.01% and 1.01%) and DTT (0.1 mM, 1.1 mM, and 10.1 mM). The starting substrate formulation was 0.15 M potassium phosphate, pH 6.5; 0.01% (w/v) Tween® 20; 0.1 mM DTT; and 0.2 mM biotin-4-MU (n-D-biotinyl-7-amino-4-methylcoumarin). To prepare a substrate formulation containing 1.01% (w/v) Tween® 20, 0.1 g of Tween® 20 was added to 10 mL of the starting substrate formulation. To prepare substrate formulations containing 1.1 mM DTT, 10 μL of a 100 mM stock of DTT was added to 990 μL of 0.01% (w/v) Tween® 20 substrate mix and to 990 μL of 1.01% (w/v) substrate mix. To prepare substrate formulations containing 10.1 mM DTT, 100 μL of a 100 mM stock of DTT was added to 900 μL of 0.01% (w/v) Tween® 20 substrate mix and to 900 μL of 1.01% (w/v) substrate mix.

Serum samples from a presumed normal individual, a biotinidase deficient sample and NEH controls were prepared as described in reference to FIGS. 1A and 1B.

The assay protocol included the following steps: An aliquot (10 μL) of fresh-frozen serum sample was mixed with 10 μL of biotin-4-MU substrate mix in separate wells of a 96-well microtiter plate. The reaction was incubated at 37° C. for 20 hours. After the incubation period, the reaction was stopped with 50 μL stop buffer (0.2 M sodium bicarbonate, pH 10.0; 0.01% (w/v) Tween® 20). Fluorescence was read at 360 nm excitation/460 nm emission at a gain of 60. Deficient (Def.), normal (Norm.), and NEH control samples were run in triplicate and an average value determined for each sample. For each deficient and normal sample, the corresponding NEH average value was subtracted. To evaluate the strength and separation of fluorescence signal between normal and deficient samples for each substrate formulation, the data was expressed as normal 4-MU fluorescence minus deficient 4-MU fluorescence. The data shows substrate formulations containing 1.01% (w/v) Tween® 20 and 1.1 mM DTT provides a stronger fluorescence signal and greater separation of fluorescence signals between normal and biotinidase deficient samples.

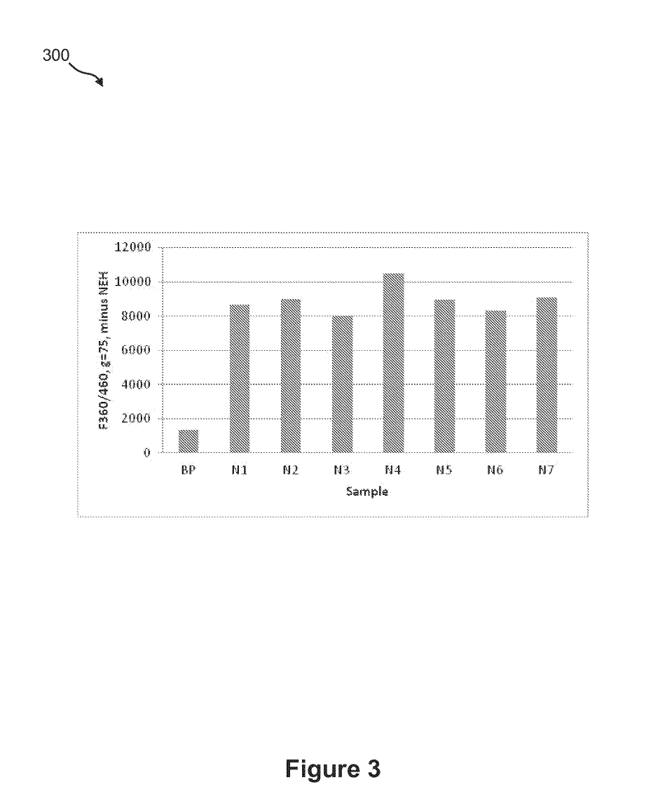

FIG. 3 shows a bar graph 300 of another example of a biotinidase enzyme activity assay performed on-bench using DBS extracts. DBS extract samples from seven presumed normal individuals and a quality control (QC) base pool (BP) dried blood sample were prepared as described herein. The dried QC-BP sample was prepared from a pool of washed, leukoreduced human red blood cells that were adjusted with human plasma to a hematocrit of 50%. The substrate formulation was 0.15 M potassium phosphate, pH 6.5; 1.01% (w/v) Tween® 20; 1.01 mM DTT; and 0.2 mM biotin-4-MU (n-D-biotinyl-7-amino-4-methylcoumarin). The assay was performed on-bench using a microtiter plate assay and plate reader.

The assay protocol included the following steps: An aliquot (10 μL) of DBS extract was mixed with 10 μL of biotin-4-MU substrate mix in separate wells of a 96-well microtiter plate. The reaction was incubated at 37° C. for 20 hours. After the incubation period, the reaction was stopped with 50 μL stop buffer (0.2 M sodium bicarbonate, pH 10.5; 0.01% (w/v) Tween® 20). For each normal DBS extract and QC-BP extract, a corresponding non-enzymatic hydrolysis (NEH) control sample was prepared as described in reference to FIG. 1. Fluorescence was read at 360 nm excitation/460 nm emission at a gain of 75, offset 1 mm. Samples were run in duplicate and an average value determined for each sample. Data is expressed as sample 4-MU fluorescence minus NEH fluorescence.

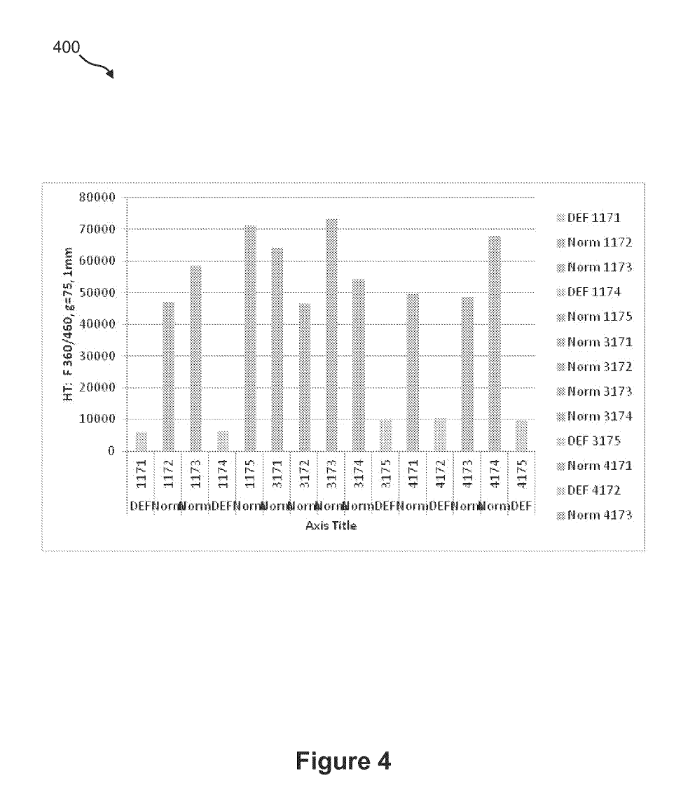

FIG. 4 shows a bar graph 400 of another example of a biotinidase activity assay performed on-bench using DBS extracts. DBS samples were obtained from U.S. Centers for Disease Control (CDC), Atlanta Ga. DBS extracts were prepared by extracting a 3 mm punch in 100 μL of extraction buffer (0.1% (w/v) Tween® 20 in molecular grade water) as described herein. The substrate formulation was 0.2 mM biotin-4-MU (n-D-biotinyl-7-amino-4-methylcoumarin); 10.1 mM DTT; 0.01% (w/v) Tween® 20; and potassium phosphate, pH 6.5. The assay was performed on-bench using a microtiter plate assay and plate reader.

The assay protocol included the following steps: An aliquot (10 μL) of DBS extract was mixed with 10 μL of biotin-4-MU substrate mix in separate wells of a 96-well microtiter plate. The reaction was incubated at 37° C. for 24 hours. After the incubation period, the reaction was stopped with 50 μL stop buffer (0.2 M sodium bicarbonate, pH 10.0; 0.01% (w/v) Tween® 20). Fluorescence was read at 360 nm excitation/460 nm emission at a gain of 75, offset 1 mm. Samples were run in duplicate. The CDC classification of biotinidase activity for each DBS sample is shown in Table 1. The data show a clear separation of fluorescence signals between normal and biotinidase deficient DBS samples.

TABLE 1
Classification of biotinidase activity for DBS samples

| | Specimen | Specimen | Specimen | Specimen | Specimen |
|---|---|---|---|---|---|
| | 1171 | 1172 | 1173 | 1174 | 1175 |
| Biotinidase | 2* | 1 | 1 | 2 | 1 |
| | Specimen | Specimen | Specimen | Specimen | Specimen |
| | 3171 | 3172 | 3173 | 3174 | 3175 |
| Biotinidase | 1 | 1 | 1 | 1 | 2 |
| | Specimen | Specimen | Specimen | Specimen | Specimen |
| | 4171 | 4172 | 4173 | 4174 | 4175 |
| Biotinidase | 1 | 2 | 1 | 1 | 2 |

*1= Normal activity 2= Deficiency

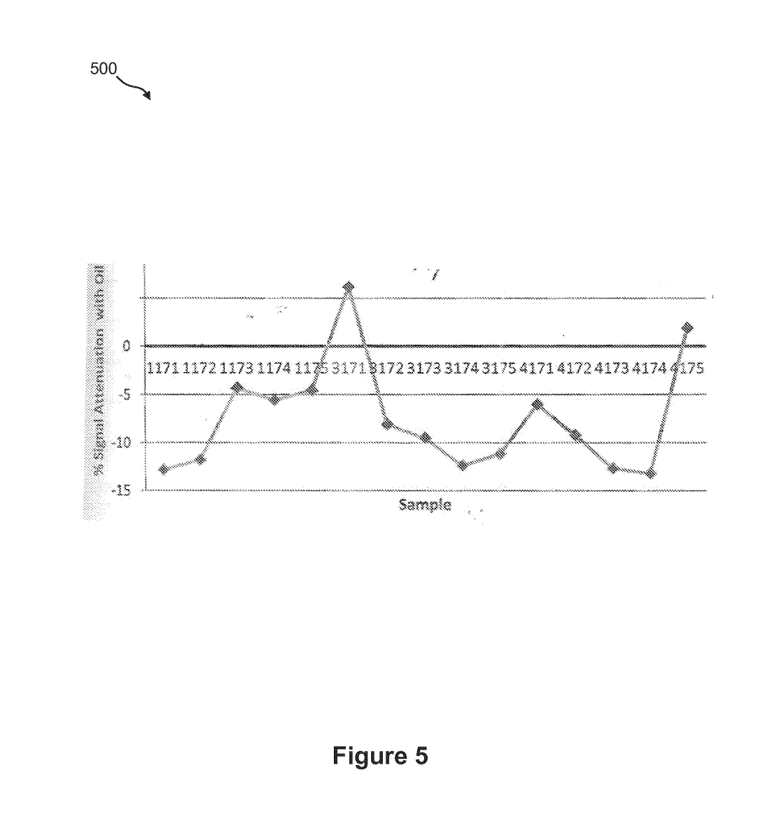

In the droplet operations environment of a droplet actuator, partitioning of 4-MU between the aqueous phase (i.e., droplet) and the organic phase (filler fluid) may potentially contaminate an electrode pathway. The enzymatic turnover of the 4-MU substrate requires a low-pH environment (acidic environment). At low pH, 4-MU is non-ionic and hydrophobic and partitions preferentially from the aqueous reaction phase into the organic phase. Reactions subsequently prepared for the detection step of the bioassay by the addition of stop buffer (0.2 M sodium bicarbonate, pH 10.0; 0.01% (w/v) Tween® 20) are at a high pH. Fluorescence of 4-MU is enhanced at elevated pH (pH >10). If 4-MU partitions into the oil phase, a decrease in fluorescence may be observed. FIG. 5 shows a plot 500 of an example of a biotinidase activity assay for evaluating partitioning of 4-MU. DBS samples, substrate formulation, and assay protocol are as described in reference to FIG. 4, except for the addition of 20 μL NBS-886-silicone oil, 0.1% Triton X-15 to the reaction mixture (reaction sample). After the incubation period, the reaction was stopped with 50 μL stop buffer and fluorescence determined. The assay was performed in parallel with the assay described in reference to FIG. 4. Data is expressed as percent signal attenuation in the presence of oil relative to fluorescence in the absence of oil (fluorescence values from FIG. 4). The data shows about 5 to 13% partitioning of 4-MU signal into the oil phase.

In various embodiments, the reaction sample is in contact with or submerged in a liquid which is immiscible with the reaction sample. The immiscible liquid may, for example, include an oil, such as a silicone oil or paraffin that is liquid at the reaction temperature, and may also include a surfactant. The surfactant may, for example, be a nonionic low hydrophile-lipophile balanced (HLB) surfactant. In some cases, the HLB of the surfactant is less than about 10 or less than about 5. Examples of suitable surfactants include Triton X-15; Span 85; Span 65; Span 83; Span 80; Span 60; and fluorinated surfactants. Various techniques may be used to substantially eliminate partitioning of 4-MU from an aqueous phase to an organic phase. In one embodiment, 4-MU-containing substrates may be retained within an aqueous phase (e.g., an aqueous droplet) by formation of an “inclusion complex”. In one example, cyclodextrins such as methyl-β-cyclodextran may be used to form an inclusion complex containing 4-MU.

### 7.1.2 Droplet-Based Assay Protocol

The invention provides methods for a droplet-based enzymatic assay for biotinidase activity. Biotinidase enzymatic activity assays may be performed using fresh blood samples, fresh-frozen blood samples or dried blood spot (DBS) samples. On-bench assays for determination of biotinidase activity may be adapted and described as discrete step-by-step droplet-based protocols.

Digital microfluidic enzyme assays are performed in aqueous droplets within an oil filled gap of a droplet actuator. Samples and assay reagents are manipulated as discrete droplets upon an arrangement of electrodes (i.e., digital electrowetting). Sample droplets and reagent droplets for use in conducting the enzymatic assays may be dispensed and/or combined according to appropriate assay protocols using droplet operations on a droplet actuator. Incubation of assay droplets, including temperature adjustments as needed, may also be performed on a droplet actuator. Further, detection of signals from assay droplets, such as detection of fluorescence may be conducted while the droplet is present on the droplet actuator. Further, each of these processes may be conducted while the droplet is partially or completely surrounded by a filler fluid on the droplet actuator.

In some embodiments, certain assay steps may be conducted outside of a droplet actuator and certain assay steps may be conducted on a droplet actuator. For example, in some embodiments, samples and reagents may be prepared outside the droplet actuator and combined, incubated and detected on the droplet actuator. In one embodiment, samples (e.g., fresh-frozen blood samples, DBS samples) used for testing for biotinidase activity may be prepared using an on-bench protocol prior to loading on a droplet actuator. Reagent preparation (e.g., extraction buffer and substrate formulations) may also be prepared using on-bench protocols prior to loading on a droplet actuator. In another embodiment reagent and/or samples may be prepared in reservoirs associated with the droplet actuator then flowed to different operations gaps, and/or prepared in the droplet operations gap.

An example of a digital microfluidic testing assay for biotinidase activity may include, but is not limited to, the following: A sample droplet (e.g., a DBS extract sample droplet) is combined and mixed using droplet operations with a biotinidase substrate droplet (e.g., 0.2 mM biotin-4-MU [n-D-biotinyl-7-amino-4-methylcoumarin], 10.1 mM DTT, 0.01% (w/v) Tween 20, and 0.15 M potassium phosphate buffer, pH 6.5) to form a reaction droplet. The reaction droplet is transported using droplet operations to a temperature control zone. The temperature control zone may, for example, be set at 36° C. to 38° C., particularly 37° C. The reaction droplet is incubated at 37° C. for a predetermined time. After the incubation period, a stop (quench) buffer droplet is dispensed and combined using droplet operations with the reaction droplet. The stop (quench) buffer includes, but is not limited to a pH buffer (e.g., sodium bicarbonate, pH 10-11) and a surfactant (e.g., a polysorbate surfactant such as Polysorbate 20/Tween® 20). The combined reaction/stop buffer droplet is transported using droplet operations to a detection spot and fluorescence measured. Biotinidase activity is determined from the fluorescence signal. In this example, a single sample droplet is dispensed and analyzed. However, any number of sample droplets may be dispensed and analyzed.

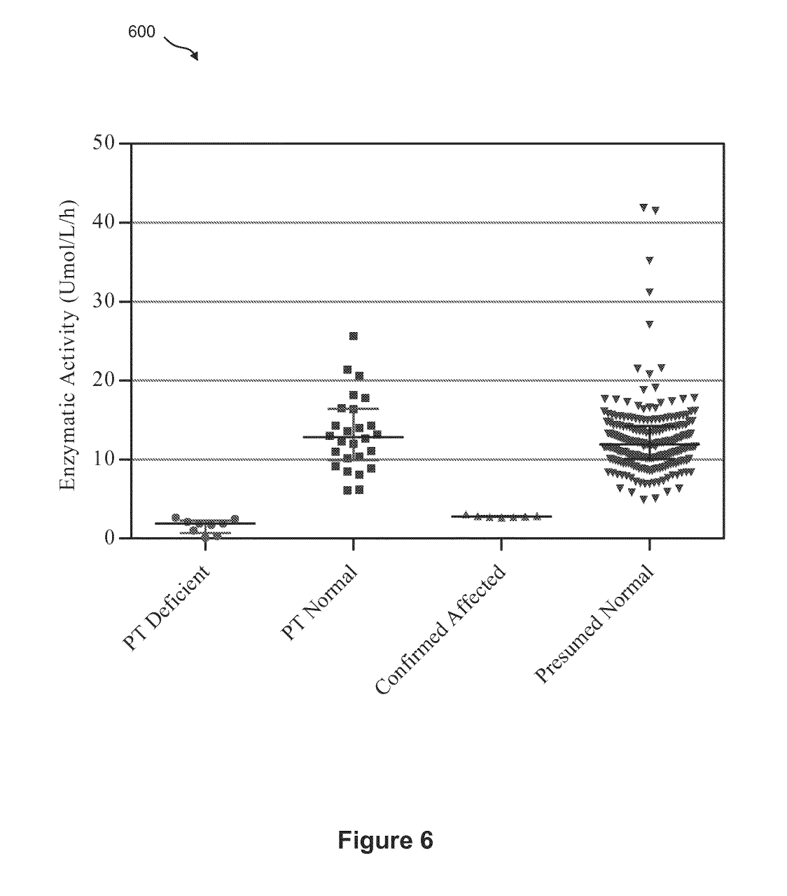

FIG. 6 shows a plot of an example of a digital microfluidic testing assay for biotinidase activity. Newborns were screened for biotinidase deficiency via a fluorimetric biotinidase enzymatic assay of DBS samples using a digital microfluidic platform. The enzymatic assay used 4-methylumbelliferyl biotin (4-MU biotin) as the fluorimetric substrate. Biotinidase deficiency screening was performed on deficient (n=9) and normal (n=26) proficiency samples and confirmed affected (n=7) and presumed normal (n=200) patient samples.

For the digital microfluidic testing assay for biotinidase activity, de-identified, presumed normal dried blood spots (NBS cards; n=200) were obtained from the North Carolina Division of Public Health NBS laboratories. These discarded specimens were 2-3 months old and were stored at −20° C. upon receipt. De-identified biotinidase affected DBS spots (n=7) were provided by the Michigan Neonatal Blood Bank. Deficient and normal biotinidase proficiency testing dried blood spots were obtained from the Centers for Disease Control and Prevention (CDC).

4-methylumbelliferyl biotin (4-MU biotin) was purchased from Toronto Research Chemicals (n-D-biotinyl-7-amino-4-methylcoumarin; cat # B394925). Sodium bicarbonate, potassium phosphate (monobasic and dibasic), DL-dithiothreitol (DTT), and Tween 20 were all obtained from Sigma-Aldrich Corp. (St. Louis, Mo.). Molecular grade water was obtained from Fisher Scientific (Pittsburgh, Pa.). 5cSt silicone oil was obtained from Gelest Inc. (Morrisville, Pa.).

Dried blood spots were punched and stored at −20° C. as follows. For each CDC proficiency sample, presumed normal (n=200) and biotinidase affected DBS (n=7), one 3 mm punch was collected in a 96-well plate. Reagent was composed of 0.1 mM 4 MU-Biotin, 1.1 mM DTT, 0.1% Tween 20 in 0.15 M KH₂PO₄, pH 6.5. Ready-to-use reagent aliquots were prepared and stored at −80° C. Extraction buffer (water with 0.1% Tween 20) and stop buffer (0.6 M NaHCO₃ pH 11.0 with 0.01% Tween 20) solutions were prepared and stored at room temperature.

Assays were performed on a disposable, single use digital microfluidic cartridge capable of accepting up to 48 samples and calibrants. This same cartridge has been used to demonstrate lysosomal storage disease screening for Pompe, Fabry, Gaucher, Hunter, and Hurler diseases (Sista et al. “Digital microfluidic platform for multiplexing enzyme assays: implications for lysosomal storage disease screening in newborns,” Clin. Chem. 57:1444-1451 (2011); Sista et al. “Rapid, single-step assay for Hunter syndrome in dried blood spots using digital microfluidics,” Clin. Chim. Acta. 412:1895-1897(2011); Tolun et al. “A novel fluorometric enzyme analysis method for Hunter syndrome using dried blood spots,” Mol. Gen. and Metab. 105:519-521 (2012); Sista et al. “Rapid assays for Gaucher and Hurler diseases in dried blood spots using digital microfluidics,” Mol. Gen. Metab. in press, available online Mar. 24, 2013 (2013)). The protocol for performing fluorimetric enzymatic assays on the digital microfluidic platform was as described by Sista et al. (Sista et al. “Digital microfluidic platform for multiplexing enzyme assays: implications for lysosomal storage disease screening in newborns,” Clin. Chem. 57:1444-1451 (2011)). Prior to the start of the assay, a four point 4-MU calibration curve was generated in duplicate on the cartridge with the following concentrations of 4-MU in stop buffer: 0.0375 μM, 0.075 μM, 0.15 μM and 0.3 μM.

The protocol for biotinidase deficiency screening using digital microfluidics was first assayed using proficiency control samples (data not shown). Biotinidase proficiency quality control samples (n=10), including seven normal and three deficient samples, were obtained from the Centers for Disease Control (CDC). One 3 mm punch was extracted from each proficiency DBS sample in 100 μL extraction buffer (0.1% (w/v) Tween 20 in water) for 30 minutes at room temperature in a 96 well plate. During this time, a digital microfluidic cartridge was inserted into the analyzer. After incubation, the DBS extract was transferred to the cartridge via a multi-channel pipette. All subsequent fluid handling operations were automated on the cartridge by the digital microfluidic platform.

To begin the reaction, one droplet (˜100 nL) of DBS extract was dispensed and mixed with one droplet (˜100 nL) of reagent (4-MU biotin at optimal pH) to form a reaction droplet (˜200 nL). The reaction droplet was incubated for one hour at 37° C. on the digital microfluidic cartridge. The reaction was stopped when a droplet of stop buffer (˜100 nL) was dispensed and merged with the reaction droplet. Endpoint fluorescence was measured at 360 nm excitation and 460 nm emission. Enzymatic activity was reported as mmoles of 4-MU produced per liter of blood per hour of incubation using the 4-MU calibration curve.

A larger study was then conducted with deficient (n=9) and normal (n=26) proficiency testing (PT) samples from the CDC, confirmed affected (n=7) samples from Michigan Neonatal Blood Bank, and presumed normal samples (n=200) from the North Carolina Division of Public Health NBS laboratories. The assay protocol was as described above. As shown in FIG. 6, the digital microfluidic platform discriminated between deficient and normal proficiency testing samples and presumed normal and known deficient patient samples samples in a fluorimetric biotinidase enzymatic assay. FIG. 6 shows plots of enzymatic activity for proficiency and patient samples. The horizontal black line represents the median and error bars represent standard deviation. Range of enzymatic activity for the presumed normal samples was 4.6-41.9 μmol/L/h and 2.29-3.2 μmol/L/h for the known affected samples. Mean enzymatic activity values were 11.7 μmol/L/h and 2.65 μmol/L/h for presumed normal samples and known affected samples respectively. No overlap in enzymatic activity was observed with clear separation between the presumed normal and affected samples.

Although the example above is directed to assaying for biotinidase enzymatic activity, the present microfluidic platform may also be used to simultaneously screen for multiple types of enzymatic assays for newborn screening, including but not limited to lysosomal storage disease screening for Pompe, Fabry, Gaucher, Hunter, and Hurler diseases. Accordingly, a single, easy to use, inexpensive, and automated platform may be used to consolidate of several assay modalities onto a single instrument for a variety of newborn screens.

### 7.2 Systems

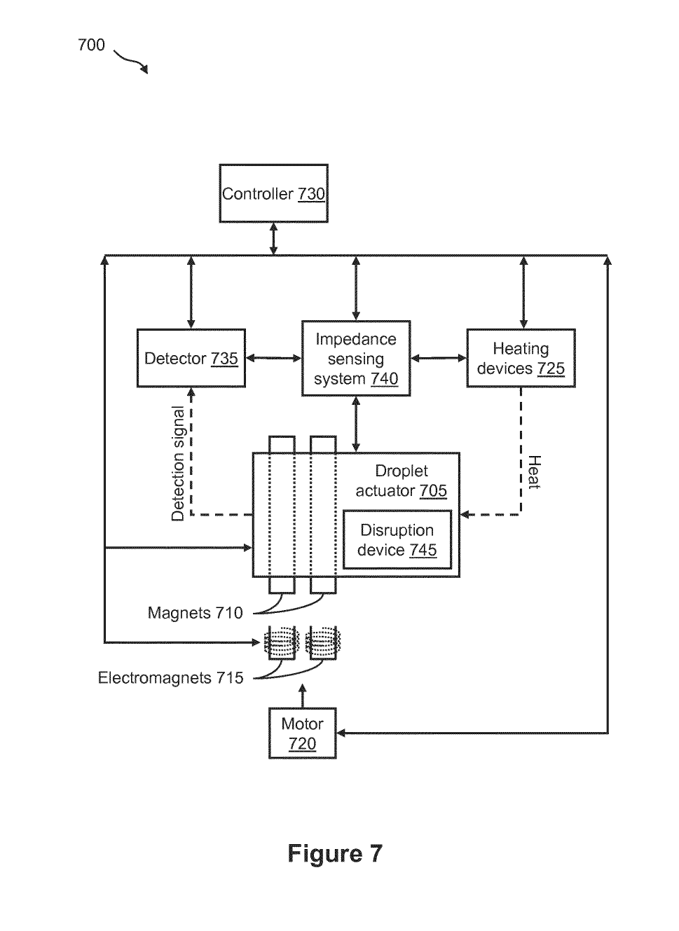

FIG. 7 illustrates a functional block diagram of an example of a microfluidics system 700 that includes a droplet actuator 705. Digital microfluidic technology conducts droplet operations on discrete droplets in a droplet actuator, such as droplet actuator 705, by electrical control of their surface tension (electrowetting). The droplets may be sandwiched between two substrates of droplet actuator 705, a bottom substrate and a top substrate separated by a droplet operations gap. The bottom substrate may include an arrangement of electrically addressable electrodes. The top substrate may include a reference electrode plane made, for example, from conductive ink or indium tin oxide (ITO). The bottom substrate and the top substrate may be coated with a hydrophobic material. Droplet operations are conducted in the droplet operations gap. The space around the droplets (i.e., the gap between bottom and top substrates) may be filled with an immiscible inert fluid, such as silicone oil, to prevent evaporation of the droplets and to facilitate their transport within the device. Other droplet operations may be effected by varying the patterns of voltage activation; examples include merging, splitting, mixing, and dispensing of droplets.

Droplet actuator 705 may be designed to fit onto an instrument deck (not shown) of microfluidics system 700. The instrument deck may hold droplet actuator 705 and house other droplet actuator features, such as, but not limited to, one or more magnets and one or more heating devices. For example, the instrument deck may house one or more magnets 710, which may be permanent magnets. Optionally, the instrument deck may house one or more electromagnets 715. Magnets 710 and/or electromagnets 715 are positioned in relation to droplet actuator 705 for immobilization of magnetically responsive beads. Optionally, the positions of magnets 710 and/or electromagnets 715 may be controlled by a motor 720. Additionally, the instrument deck may house one or more heating devices 725 for controlling the temperature within, for example, certain reaction and/or washing zones of droplet actuator 705. In one example, heating devices 725 may be heater bars that are positioned in relation to droplet actuator 705 for providing thermal control thereof.

A controller 730 of microfluidics system 700 is electrically coupled to various hardware components of the invention, such as droplet actuator 705, electromagnets 715, motor 720, and heating devices 725, as well as to a detector 735, an impedance sensing system 740, and any other input and/or output devices (not shown). Controller 730 controls the overall operation of microfluidics system 700. Controller 730 may, for example, be a general purpose computer, special purpose computer, personal computer, or other programmable data processing apparatus. Controller 730 serves to provide processing capabilities, such as storing, interpreting, and/or executing software instructions, as well as controlling the overall operation of the system. Controller 730 may be configured and programmed to control data and/or power aspects of these devices. For example, in one aspect, with respect to droplet actuator 705, controller 730 controls droplet manipulation by activating/deactivating electrodes.

In one example, detector 735 may be an imaging system that is positioned in relation to droplet actuator 705. In one example, the imaging system may include one or more light-emitting diodes (LEDs) (i.e., an illumination source) and a digital image capture device, such as a charge-coupled device (CCD) camera.

Impedance sensing system 740 may be any circuitry for detecting impedance at a specific electrode of droplet actuator 705. In one example, impedance sensing system 740 may be an impedance spectrometer. Impedance sensing system 740 may be used to monitor the capacitive loading of any electrode, such as any droplet operations electrode, with or without a droplet thereon. For examples of suitable capacitance detection techniques, see Sturmer et al., International Patent Publication No. WO/2008/101194, entitled “Capacitance Detection in a Droplet Actuator,” published on Aug. 21, 2008; and Kale et al., International Patent Publication No. WO/2002/080822, entitled “System and Method for Dispensing Liquids,” published on Oct. 17, 2002; the entire disclosures of which are incorporated herein by reference.

Droplet actuator 705 may include disruption device 745. Disruption device 745 may include any device that promotes disruption (lysis) of materials, such as tissues, cells and spores in a droplet actuator. Disruption device 745 may, for example, be a sonication mechanism, a heating mechanism, a mechanical shearing mechanism, a bead beating mechanism, physical features incorporated into the droplet actuator 705, an electric field generating mechanism, a thermal cycling mechanism, and any combinations thereof. Disruption device 745 may be controlled by controller 730.

It will be appreciated that various aspects of the invention may be embodied as a method, system, computer readable medium, and/or computer program product. Aspects of the invention may take the form of hardware embodiments, software embodiments (including firmware, resident software, micro-code, etc.), or embodiments combining software and hardware aspects that may all generally be referred to herein as a “circuit,” “module” or “system.” Furthermore, the methods of the invention may take the form of a computer program product on a computer-usable storage medium having computer-usable program code embodied in the medium.

Any suitable computer useable medium may be utilized for software aspects of the invention. The computer-usable or computer-readable medium may be, for example but not limited to, an electronic, magnetic, optical, electromagnetic, infrared, or semiconductor system, apparatus, device, or propagation medium. The computer readable medium may include transitory and/or non-transitory embodiments. More specific examples (a non-exhaustive list) of the computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Program code for carrying out operations of the invention may be written in an object oriented programming language such as Java, Smalltalk, C++ or the like. However, the program code for carrying out operations of the invention may also be written in conventional procedural programming languages, such as the “C” programming language or similar programming languages. The program code may be executed by a processor, application specific integrated circuit (ASIC), or other component that executes the program code. The program code may be simply referred to as a software application that is stored in memory (such as the computer readable medium discussed above). The program code may cause the processor (or any processor-controlled device) to produce a graphical user interface (“GUI”). The graphical user interface may be visually produced on a display device, yet the graphical user interface may also have audible features. The program code, however, may operate in any processor-controlled device, such as a computer, server, personal digital assistant, phone, television, or any processor-controlled device utilizing the processor and/or a digital signal processor.

The program code may locally and/or remotely execute. The program code, for example, may be entirely or partially stored in local memory of the processor-controlled device. The program code, however, may also be at least partially remotely stored, accessed, and downloaded to the processor-controlled device. A user's computer, for example, may entirely execute the program code or only partly execute the program code. The program code may be a stand-alone software package that is at least partly on the user's computer and/or partly executed on a remote computer or entirely on a remote computer or server. In the latter scenario, the remote computer may be connected to the user's computer through a communications network.

The invention may be applied regardless of networking environment. The communications network may be a cable network operating in the radio-frequency domain and/or the Internet Protocol (IP) domain. The communications network, however, may also include a distributed computing network, such as the Internet (sometimes alternatively known as the “World Wide Web”), an intranet, a local-area network (LAN), and/or a wide-area network (WAN). The communications network may include coaxial cables, copper wires, fiber optic lines, and/or hybrid-coaxial lines. The communications network may even include wireless portions utilizing any portion of the electromagnetic spectrum and any signaling standard (such as the IEEE 802 family of standards, GSM/CDMA/TDMA or any cellular standard, and/or the ISM band). The communications network may even include powerline portions, in which signals are communicated via electrical wiring. The invention may be applied to any wireless/wireline communications network, regardless of physical componentry, physical configuration, or communications standard(s).

Certain aspects of invention are described with reference to various methods and method steps. It will be understood that each method step can be implemented by the program code and/or by machine instructions. The program code and/or the machine instructions may create means for implementing the functions/acts specified in the methods.

The program code may also be stored in a computer-readable memory that can direct the processor, computer, or other programmable data processing apparatus to function in a particular manner, such that the program code stored in the computer-readable memory produce or transform an article of manufacture including instruction means which implement various aspects of the method steps.

The program code may also be loaded onto a computer or other programmable data processing apparatus to cause a series of operational steps to be performed to produce a processor/computer implemented process such that the program code provides steps for implementing various functions/acts specified in the methods of the invention.

### Concluding Remarks

The foregoing detailed description of embodiments refers to the accompanying drawings, which illustrate specific embodiments of the invention. Other embodiments having different structures and operations do not depart from the scope of the present invention. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

1. A method of conducting an assay for biotinidase, the method comprising:
(a) providing a sample;
(b) providing a substrate formulation, wherein the substrate formulation comprises a substrate that releases a fluorophore upon contact with biotinidase;
(c) mixing the sample with the substrate formulation to produce a reaction sample;
(d) incubating the reaction sample;
(e) mixing a stop buffer with the reaction sample; and
(f) measuring a fluorescence signal in the reaction sample, wherein the fluorescence signal correlates with the presence and/or activity of biotinidase in the sample.

2. The method of claim 1, wherein the sample comprises fresh blood, fresh-frozen blood, plasma, fresh-frozen plasma, or a dried blood spot.

3. The method of claim 2, wherein the fresh blood, fresh-frozen blood, plasma, fresh-frozen plasma, or dried blood spot is from a newborn infant.

4. The method of claim 2, wherein the fresh blood, fresh-frozen blood, plasma, fresh-frozen plasma, or dried blood spot is mixed with an extraction buffer.

5. The method of claim 4, wherein the extraction buffer comprises a surfactant.

6. The method of claim 5, wherein the surfactant is a polysorbate surfactant.

7. The method of claim 6, wherein the polysorbate surfactant is Polysorbate 20.

8. The method of claim 1, wherein the substrate formulation further comprises an assay buffer.

9. The method of claim 8, wherein the assay buffer comprises a pH buffer, a reducing agent, and a surfactant.

10. The method of claim 9, wherein the pH buffer is a potassium phosphate buffer.

11. The method of claim 10, wherein the potassium phosphate buffer is about 6.5 pH.

12. The method of claim 9, wherein the reducing agent is dithiothreitol (DTT).

13. The method of claim 9, wherein the surfactant is a polysorbate surfactant.

14. The method of claim 13, wherein the polysorbate surfactant is Polysorbate 20.

15. The method of claim 1, wherein the substrate that releases a fluorophore upon contact with biotinidase is a biotin-4-MU fluorescent substrate.

16. The method of claim 15, wherein the biotin-4-MU fluorescent substrate is n-D-biotinyl-7-amino-4-methylcoumarin.

17. The method of claim 16, wherein measuring a fluorescence signal comprises reading fluorescence at 360 nm excitation and 460 nm emission.

18. The method of claim 16, wherein the biotin-4-MU fluorescent substrate is dissolved in a solvent before addition to the substrate formulation.

19. The method of claim 18, wherein the solvent is a polar aprotic solvent.

20. The method of claim 19, wherein the polar aprotic solvent is dimethyl sulfoxide (DMSO).

21. The method of claim 1, wherein the reaction sample is incubated at between 36° C. and 38° C.

22. The method of claim 21, wherein the reaction sample is incubated at about 37° C.

23. The method of claim 1, wherein the reaction sample is incubated for up to 24 hours.

24. The method of claim 1, wherein the reaction sample is incubated for up to 20 hours.

25. The method of claim 1, wherein the reaction sample is incubated for up to 1 hour.

26. The method of claim 1, wherein the stop buffer comprises a pH buffer and a surfactant.

27. The method of claim 26, wherein the pH buffer is a sodium bicarbonate buffer.

28. The method of claim 27, wherein the sodium bicarbonate buffer is between pH 10.0-11.0.

29. The method of claim 26, wherein the surfactant is a polysorbate surfactant.

30. The method of claim 29, wherein the polysorbate surfactant is Polysorbate 20.

31. The method of claim 1, wherein any of the steps are performed in one or more droplets in oil.

32. The method of claim 31, wherein the oil is silicone oil.

33. The method of claim 1, wherein the sample is loaded onto a droplet actuator and the method is performed by executing droplet operations on the droplet actuator.

34. The method of claim 33, wherein the droplet actuator comprises a filler fluid comprising an oil.

35. The method of claim 34, wherein the filler fluid comprises a silicone oil.

36. The method of claim 34, wherein the filler fluid further comprises a nonionic low hydfophile-Iipophile balanced (HLB) surfactant.

37. The method of claim 33, wherein the droplet operations comprise electrode-mediated droplet operations.

38. The method of claim 33, wherein the droplet operations are eicefrowerting mediated.

39. The method of claim 33, wherein the droplet operations are dielectrophoresis mediated.

40. A computer readable medium programmed to cause a droplet actuator to perform any of the steps of claim 1.

41. A system comprising a droplet actuator coupled to and controlled by a computer programmed to cause the droplet actuator to perform any of the steps of claim 1.
